(19) United States
(12) Patent Application Publication
(10) Pub. No.: US 2011/0118462 A1
(43) Pub. Date: May 19, 2011

(54) N-HETEROCYCLIC SUBSTITUENT-CONTAINING ANTIBIOTIC, PREPARATION AND USE THEREOF

(75) Inventors: Mao CHEN, Guangzhou City (CN); Shaoxuan Zhu, Guangzhou City (CN); Xuebin Liu, Guangzhou City (CN); Lizhen Zheng, Guangzhou City (CN); Liwei Lin, Guangzhou City (CN); Shuwen Xu, Guangzhou City (CN); Yuping Wang, Guangzhou City (CN); Wei Yang, Guangzhou City (CN); Yunfeng Li, Guangzhou City (CN); Fang Ye, Guangzhou City (CN); Xiaona Zhang, Guangzhou City (CN)

(73) Assignees: GUANGZHOU BAIYUNSHAN PHARMACEUTICAL CO., LTD. GUANGZHOU BAIYUNSHAN PHARMACEUTICAL FACTORY, Guangzhou (CN); GUANGZHOU PHARMACEUTICAL INDUSTRIAL RESEARCH INSTI, Guangzhou (CN)

(21) Appl. No.: 12/621,237

(22) Filed: Nov. 18, 2009

(51) Int. Cl. C07D 501/16

(52) U.S. Cl. 544/48

(57) ABSTRACT

The invention relates to N-heterocyclic substituent-containing antibiotics, their preparation, and their use. Disclosed are sodium and potassium salts of 7-(α-((N,N′-diisopropylamidino)thio)acetylamino)-3-(((1,2,5,6-tetrahydro-2-methyl-5,6-dioxo-1,2,4-triazin-3-yl)thio)methyl) cephalosporanic acid as presented by the general structure (I), their preparation, and their use. The antibiotics of the invention can be used to treat diseases caused by Gram-positive or Gram-negative bacteria such as septicaemia, gastrointestinal tract infection, and urinary tract infection. They have increased half-life in blood and lowered toxicity. They can reduce the frequency of drug use and lower medical treatment costs. They have improved stability and can be stored at ambient temperatures. The method of the invention is simple, and it produces high purity products which can meet the requirements of clinical use.

### FIELD OF THE INVENTION

The invention relates to N-heterocyclic substituent-containing antibiotics. More particularly, the invention relates to sodium or potassium salts of 7-(α-((N,N′-diisopropyl amidino)thio)acetylamino)-3-(((1,2,5,6-tetrahydro-2-methyl-5,6-dioxo-1,2,4-triazin-3-yl)thio) methyl) cephalosporanic acid, their preparation, and their uses.

### BACKGROUND OF THE INVENTION

Ceftriamidine, chemical name 7-(α-((N,N′-diisopropylamidino)thio)acetylamino)-3-(((1,2,5,6-tetrahydro-2-methyl-5,6-dioxo-1,2,4-triazin-3-yl)thio)methyl) cephalosporanic acid is a new cephalosporin compound. Its structure is:

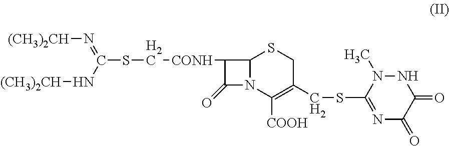

Compound II was first disclosed in Chinese Patent Application No. 200410050908. It is a cefathiamidine type compound and has relatively high antibacterial activity against certain Gram-positive bacteria.

We found that Compound II has a relatively low solubility in water and a low stability. It has a pH value of 3.2, and it irritates muscles and blood vessels. It is not suitable for direct medical use.

Because the amidinothio-group is basic, it readily forms zwitterion with the carboxyl group on position 2 of cephalosporin. Therefore, a formamidine-containing cephalosporin exists as a zwitterion. According to literature, Compound II disclosed in Chinese Patent 200410050908.X can exist as a free acid, a hydrobromide salt, or amine salt. Cefathiamidine, which has a similar structure, also forms an inner amine salt with carboxylic acid on position 2 (see Pharmacopoeia of the People's Republic of China, 2^{nd} Edition, page 156). Wang Wen-Mei reported (see Pharmaceutical Journal, Vol. 16(7), page 494 (1981)) that 7-bromoacetamido cephalosporin derivatives react with various thiourea derivatives to form cephalosporin derivatives which can exist as zwitterions, free acids, or hydrobromide salts. Chinese Invention. Pat. No. 03113688.5 also disclosed the preparation of cephalosporin amine salts and used them for the purification of cefathiamidine. However, no reference teaches the preparation of hydrobromide salts. In summary, the current research relating to amidinothio-containing cephalosporin deals only with the chemical structures of its zwitterions, free acids, hydrobromide salts and amine salts.

According to the synthesis method disclosed in Chinese Pat. No. 200410050908.X, the inventors here conducted further study. They found that due to the excess amount of organic base, e.g., triethylamine, used during the reaction, the resultant amidinothio-containing cephalosporin contains triethylamine, and thus the triethylamine salt of structure II readily absorbs moisture which is difficult to remove. Because Compound II contains triethylamine salt, it has reduced purity, darkened color, and high toxicity. Although Chinese Pat. No. 200410050908.X mentions that Compound II can exsit as an acid, a hydrobromide salt or an amine salt, it does not teach how to prepare the hydrobromide salt or amine salt.

Chinese Pat. No. 200410050908.X discloses the detailed preparation method of ceftriamidine: 7-bromine acetyl ACT reacts with N,N′-diisopropylthiourea in alkyl halides, ketones, alcohols, or mixtures thereof. As indicated in the patent specification pages 6-7, the purity can reach up to 90%. A higher purity can be obtained using acid and base to adjust the isoelectric point. However the use of acid and base can break the β-lactam ring, reduce the product purity, and reduce the product stability. The solvent-crystal separation method can likewise be used. Nevertheless, some impurities cannot be removed.

### DETAILED DESCRIPTION OF THE INVENTION

One objective of the invention is to provide an N-heterocyclic substituent-containing antibiotic. Another objective of the invention is to provide a simple method to prepare high purity ceftriamidine alkali metal salts. Still another objective of the invention is to provide a drug composition which contains ceftriamidine alkali metal salts as an active ingredient. The invention is a N-heterocyclic substituent-containing antibiotic, including sodium or potassium salts of 7-(α-((N,N-diisopropylamidino)thio)acetylamino)-3-(((1,2,5,6-tetrahydro-2-methyl-5,6-dioxo-1,2,4-triazin-3-yl)thio)methyl) cephalosporanic acid having the following general structures:

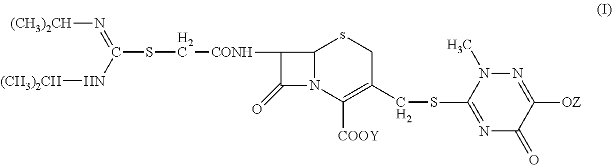

wherein:

Ia: Y=H, Z=Na;

Ib: Y=Na, Z=Na;

Ic: Y=H, Z=K;

Id: Y=K, Z=K.

A preferred embodiment has the structure of Ia:

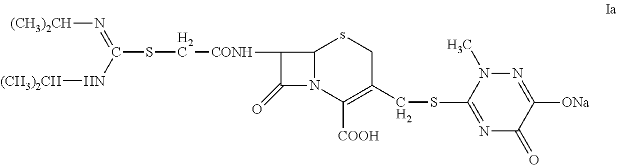

Its chemical name is 7-(α-((N,N-diisopropylamidino)thio)acetylamino)-3-(((1,2,5,6-tetrahydro-2-methyl-5,6-dioxo-1,2,4-triazin-3-yl)thio)methyl) cephalosporanic acid mono sodium salt (Compound Ia). It is readily soluble in water and methanol but is not readily soluble in anhydrous ethanol or acetone.

Amidinothio-containing cephalosporins, such as Compound II, are known to exist as zwitterions, free acids, hydrobromide salts or amine salts. The inventors believe, through analyzing the structure feature, that besides the acidity from the carboxylic acid on the position 2, the triazine ring on the position 3 can undergo keto-enol tautomerism and exhibits acidity, as shown in the following equation:

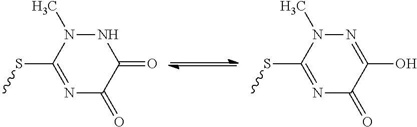

Thus, even after ceftriamidine forms zwitterions, it can still form alkali metal salts and even di-metal salts. The inventors performed a number of experiments to study the synthetic route of these salts, successfully prepared the alkali metal salts of Compound II, and thus proved their original conception. The inventors also conducted pharmaceutical study and surprisingly found that the alkali metal salts of Compound II have greater application value than ceftriamidine. It has increased water solubility and does not irritate. The preliminary pharmacokinetic study on Beagle dogs indicates that the alkali metal salts of Compound II has a biological half-life in blood of 2.59 hours which is four times longer than that of cefathiamidine (0.65 hour). It thus can reduce the frequency of drug use and increase patients' drug compliance. In addition, compared with ceftriamidine, its alkali metal salts have improved antibacterial activity, increased half-life in blood, lowered toxicity, and improved stability; they reduce frequency of use and medical treatment costs. It can be used to treat many diseases caused by Gram-positive and Gram-negative bacteria such as respiratory infection, wound and surgical infection, urinary tract infection, ear, nose and throat infection, meningitis, pleurisy, endocardium inflammation, septicaemia, lungs infection and gastrointestinal tract infection. It can be directly used and is of great interest in clinical use. It is a new cephalosporin which is of future development.

The preparation of the sodium or potassium salts of 7-(α-((N,N-diisopropylamidino)thio) acetylamino)-3-(((1,2,5,6-tetrahydro-2-methyl-5,6-dioxo-1,2,4-triazin-3-yl)thio)methyl) cephalosporanic acid as shown in general structure I is as follows:

1. Dissolving or suspending 7-(α-((N,N′-diisopropylamidino)thio)acetyl amino)-3-(((1,2,5,6-tetrahydro-2-methyl-5,6-dioxo-1,2,4-triazin-3-yl)thio)methyl) cephalosporanic acid in a solvent system and reacting it with a sodium-containing or potassium-containing alkali compound.

Suitable solvents are selected from water, alkyl ketones, alcohols, nitriles, amides, or mixtures thereof. The alkyls are C₁-C₁₂, preferably C₁-C₄ alkyls.

The sodium-containing alkali compound is selected from C₁-C₅ sodium alkoxides, sodium aliphatic or aromatic carboxylates, or inorganic sodium salts. Such as sodium methoxide, sodium acetate, sodium 2-ethylhexanoate, sodium tartrate, sodium hydroxide, sodium carbonate, and sodium bicarbonate. The potassium-containing alkali compound is selected from potassium hydroxide, potassium carbonate, and potassium bicarbonate.

In the above reaction, when the ratio of the sodium-containing or potassium-containing alkali compound to 7-(α-((N,N′-diisopropylamidino)thio)acetylamino)-3-(((1,2,5,6-tetrahydro-2-methyl-5,6-dioxo-1,2,4-triazin-3-yl)thio)methyl) cephalosporanic acid is 1:1, its mono-metal salt is formed; when the ratio is 2:1, its di-metal salt is formed such as di-sodium salt or di-potassium salt.

2. Precipitating and isolating the product of step 1.

The precipitating agents are selected from C₁-C₄ alcohols, C₃-C₆ ketones, or mixtures thereof.

In the salt forming reaction, Compound II is dissolved in water. Due to relatively strong acidity, Compound II will decompose rapidly, resulting in a low purity. Therefore, in the sodium or potassium transfer reaction, Compound II should be quickly dispersed in a solvent, such as ethanol or acetonitrile, at an agitation rate of 200-500 rpm and at a temperature below 40° C. The reaction is monitored by an on-line particle analyzing instrument. Otherwise, adding the precipitation agent, acetone or ethanol, to the reaction mixture produces only oily product and no solid product will be precipitated.

Vitro bacteriostasis experiments of Compounds Ia, Ib, and Ic were performed using the two-fold agar dilution method. Surprisingly, we found that novel Compound I not only has antibacterial activity to Gram-positive bacteria but also has relatively good antibacterial activity to Gram-negative bacteria (see Table 1).

TABLE 1
Antibacterial Test Results for
Compounds Ia, Ib, and Ic MIC₉₀ (mg · L⁻¹)

| | Compound | Compound | Compound |
|---|---|---|---|
| Bacteria | Ia | Ib | Ic |
| Excrement Enterococcus | 8 | 4 | 8 |
| Type A Chain Coccus | 0.5 | 0.1 | 0.1 |
| Type B Chain Coccus | 0.0625 | 0.0125 | 0.0156 |
| Pneumonia Chain Coccus | 0.125 | 0.5 | 0.5 |
| Haemophilus Influenzae | 0.5 | 0.5 | 0.5 |
| Moraxella Catarrhalis | 1 | 0.5 | 0.5 |
| Shigella | 4 | 4 | 4 |

The inventors conducted preliminary pharmacokinetic study of Compound Ia on Beagle dogs. The Beagle dogs were allowed to drink water freely but not to eat food for 16 hours before and 4 hours after the intravenous injection of 50 mg/kg·bw of the compound. Intravenous blood sampling took place at 0, 0.083, 0.17, 0.25, 0.5, 1, 1.5, 2, 3, 4, 6, 9, 12 hours, respectively, after the injection, each blood sample being 3 mL. Thereafter the dogs were given the drugs every other week and blood samples were taken according to the above manner. The blood samples were added to the centrifuge tubes containing heparin and thoroughly mixed by centrifuging at 4000 rpm for 10 minutes. The blood plasma was separated and an abstraction agent (methanol/ethyl acetate 3:2 V/V) was added to and mixed with it by centrifuging at 10000 rpm for 10 minutes. The top, clear liquid, 20 μL, was taken and analyzed by HPLC. The results indicate that for healthy Beagle dogs, after a single administration of Ia (50 mg/kg·bw), the Cmax is 116.35±12.06 μg/mL; after 12 hours of the administration, the drug concentration remains as high as 0.39±0.09 μg/mL; t1/2α is 0.63±0.12 h, indicating a quick distribution, t1/2β is 2.59±0.04 h, significantly longer than that of cefathiamidine which is 0.65±0.03 h; AUC is 108.29±18.17 mg/L·h, V1 is 0.25±0.07 L/kg, VB is 1.61±0.30 L/kg, indicating a broad distribution; CIB is 047±0.09 L/kg/h. Acute toxicity tests on rats indicate that LD₅₀ of Ia is 1.7476 g/kg for intravenous injection, exhibiting less toxicity as compared with cefathiamidine (which LD₅₀ is 0.8242 g/kg). In-vivo antibacterial activity of Ia has been studied; it offers excellent protection for rats against Excrement Enterococcus, Pneumonia Chain Coccus, and Moraxella Catarrhalis.

The inventors have conducted the muscle stimulation test of Compounds Ia and II on rabbits. The test is as follows. Eight New Zealand rabbits were divided into three groups. Two groups, containing three rabbits per group, were administrated with Compound Ia and Compound II, respectively. The third group, containing two rabbits, was used as a negative control group. The rabbits were intramuscularly injected with high concentrations of the compounds into the left quadriceps and with low concentration into the right quadriceps. The negative control group was injected with an equal concentration of sodium chloride into the quadriceps of both sides. Each quadriceps was administrated with 1.0 mL once a day for three consecutive days. One or two rabbits from each group were anatomized after 48 hours of the injection. The remaining rabbits were anatomized after the end of the two-week recovery period. After 48 hours and 14 days of the last drug administration, one rabbit injected with Compound Ia was observed to have slight irritation while various levels of intense irritation was observed in those rabbits injected with Compound II.

As shown above, Compound Ia has good antimicrobial activity, long half-life in blood, and low toxicity; it can reduce the frequency of drug use and lower medical cost. It is a new cephalosporin which will be developed in the future. It can be used to treat diseases caused by Gram-positive and Gram-negative bacteria such as respiratory infection, wound and surgical infection, urinary tract infection, ear, nose and throat infection, meningitis, pleurisy, endocardium inflammation, septicaemia, lungs infection and gastrointestinal tract infection.

Compared to Compound II, Compound I has better stability in aqueous solution (see Table 2) and in the solid state (See Table 3). Thus Compound I can be stored without freezing or special treatment. From a commercial point of view, the high stability of Compound I is significantly advantageous.

TABLE 2
Change of contents of Compounds Ia, Ib and II
in aqueous injection solutions at 37° C.

| | Time | | | | | |
|---|---|---|---|---|---|---|
| Compound | 0 hr | 0.2 hr | 1 hr | 2 hr | 3 hr | 4 hr |
| II | 98.50% | 94.33% | 84.26% | 77.04% | 71.06% | 66.48% |
| Ia | 98.60% | 98.60% | 97.98% | 97.79% | 97.24% | 97.13% |
| Ib | 98.80% | 98.54% | 97.14% | 96.66% | 95.21% | 94.30% |

TABLE 3
Change of contents of Compounds II and Ia after 72 hours in
storage at various temperatures

Temperature

| | −5° C. | 10° C. | 40° C. | 60° C. |
|---|---|---|---|---|
| II content, % | 98.0 | 97.9 | 96.3 | 92.75 |
| Ia content, % | 97.0 | 96.9 | 96.6 | 96.4 |

Following the disclosure of the invention involving the method for preparing alkali metal salts of N-heterocyclic substituent-containing antibiotic, the technical persons skilled in the art may make changes to the raw materials, process parameters, and structure designing, etc. The products and processes of this invention have been exemplified by the preferred examples. Skilled persons in the art may obviously make minor changes or rearrangements which fall within the spirit of the invention. Specifically, any substitution or improvement which is obvious to a person of ordinary skill in the art belongs completely to this invention.

The invention provides an antibiotic which has improved antibacterial activity and can be used to treat diseases caused by sensitive Gram-positive and Gram-negative bacteria, such as septicaemia, and lungs, gastrointestinal tract and urinary tract infections. It has a long half-life in blood and low toxicity; it can reduce the frequency of drug use and lower medical cost. The compound of the invention has improved stability and it can be stored at ambient temperatures. The preparation method of the invention can be readily practiced and the resulting products have high purity.

### EXAMPLES

### Example 1

In the following example, 7-bromine acetyl ACT refers to: 7-bromoacetamido-3-(((1,2,5,6-tetrahydro-2-methyl-5,6-dioxo-1,2,4-triazin-3-yl)thio)methyl) cephalosporanic acid.

1. 7-(α-((N,N′-diisopropylamidino)thio)acetylamino)-3-(((1,2,5,6-tetrahydro-2-methyl-5,6-dioxo-1,2,4-triazin-3-yl)thio)methyl) cephalosporanic acid (Compound II) is prepared according to Chinese invention patent 200410050908.X, entitled “Cephalosporins Having A C3 N-Containing Heterocyclic Substituted Methyl Group And A Amidinothio Acetamido Group, Preparations And Applications Thereof.”

Methylene chloride (100 mL) and 7-bromine acetyl ACT (4.9 g, 0.01 mol) were added to a three-neck flask; triethylamine (2.8 mL, 0.02 mol) was added to the flask dropwise to dissolve the solid; and N,N′-diisopropylthiourea (2.4 g, 0.015 mol) was then added to the flask. The reaction was performed at 30° C. to completion. The reaction mixture was stirred for an hour, cooled for an hour, and then filtered. The solid product was dried under vacuum to yield 4 g of Compound II having 94.3% purity by HPLC.

The pH value of its saturated aqueous solution is 3.2.

¹HNMR (DMSO-d₆, 400 Hz): 1.16 (m, 12H, J=6), 3.34 (d, 1H, J=17.5), 3.63 (d, 1H, J=17.5), 3.57 (s, 3H), 3.94 (m, 4H), 4.13 (d, 1H, J=13), 4.33 (d, 1H, J=13), 5.04 (d, 1H, J=5), 5.57 (q, 1H), 9.42 (d, 1H, J=7.5)

IR (KBr, cm⁻¹): 1783, 1657, 1606, 1411, 1339

m/e: 572

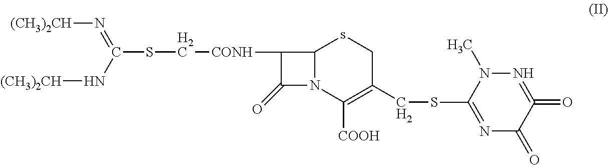

2. Preparation of 7-(α-((N,N′-diisopropylamidino)thio)acetylamino)-3-(((1,2,5,6-tetrahydro-2-methyl-5,6-dioxo-1,2,4-triazin-3-yl)thio)methyl) cephalosporanic acid monosodium salt (Compound Ia).

At 0° C., a three-neck flask was charged with deionized water (5 mL), sodium 2-ethylhexanoate (0.3 g), Compound II (1 g), and acetone (5 mL). The mixture was agitated at 350 rpm for 45 minutes, discolored with activated carbon, and filtered. The filtration liquid was dropwise added to 60 mL of acetone to precipitate. The precipitation was monitored by an on-line particle analyzing instrument. The mixture was filtered and the solid was washed twice with acetone. Drying under vacuum at 40° C. (phosphorus pentoxide) gave 0.75 g of Compound Ia with a purity of 98.76% by HPLC.

¹HNMR (DMSO-d₆, 500 Hz): 1.13 (m, 12H, J=6), 3.34 (d, 1H, J=17.5), 3.56 (d, 1H, J=17.5), 3.51 (s, 3H), 3.87 (m, 4H), 4.15 (d, 1H, J=13), 4.32 (d, 1H, J=13), 4.99 (d, 1H, J=5), 5.50 (q, 1H), 9.35 (d, 1H, J=7.5)

IR (KBr, cm⁻¹): 1767, 1605, 1498, 1403, 1366

m/e: 594

Elemental analysis: C₂₁H₂₈N₇NaO₆S₃

| | Calculated value | Measured value |
|---|---|---|
| C: | 42.45% | 42.39% |
| H: | 4.72% | 4.94% |
| N: | 16.51% | 16.45% |
| S: | 16.17% | 16.13% |

Sodium Ion Chromatography:

| | Calculated value | Measured value |
|---|---|---|
| Na: | 3.87% | 3.99% |

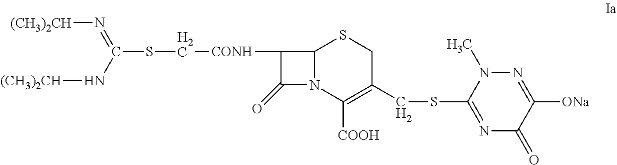

### Example 2

1. The preparation of 7-(α-((N,N′-diisopropylamidino)thio)acetylamino)-3-(((1,2,5,6-tetrahydro-2-methyl-5,6-dioxo-1,2,4-triazin-3-yl)thio)methyl) cephalosporanic acid (Compound II) is the same as in Example 1.

2. Preparation of 7-(α-((N,N′-diisopropylamidino)thio)acetylamino)-3-(((1,2,5,6-tetrahydro-2-methyl-5,6-dioxo-1,2,4-triazin-3-yl)thio)methyl) cephalosporanic acid monosodium salt (Compound Ia).

At 5° C., sodium carbonate (0.1 g) was dissolved in deionized water (5 mL) in a three-neck flask. Compound II (1 g) was added to the flask with agitation (350 rpm) and the reaction was carried out for 45 minutes. The reaction mixture was discolored with activated carbon and then filtered. The filtration liquid was dropwise added to 80 mL of acetone to precipitate. The precipitation was monitored by an on-line particle analyzing instrument. The mixture was filtered and the solid was washed twice with acetone. Drying under vacuum at 40° C. for 24 hours gave 0.75 g of Compound Ia with a purity of 98.35% (HPLC) and pH 7.3 (concentration 0.1 g/mL).

¹HNMR (DMSO-d₆, 500 Hz): 1.12 (m, 12H, J=6), 3.35 (d, 1H, J=17.5), 3.57 (d, 1H, J=17.5), 3.50 (s, 3H), 3.77 (m, 4H), 4.14 (d, 1H, J=13), 4.37 (d, 1H, J=13), 4.99 (d, 1H, J=5), 5.50 (q, 1H), 9.32 (d, 1H, J=7.5).

### Example 3

1. The preparation of 7-(α-((N,N′-diisopropylamidino)thio)acetylamino)-3-(((1,2,5,6-tetrahydro-2-methyl-5,6-dioxo-1,2,4-triazin-3-yl)thio)methyl) cephalosporanic acid (Compound II) is the same as in Example 1.

2. Preparation of 7-(α-((N,N′-diisopropylamidino)thio)acetylamino)-3-(((1,2,5,6-tetrahydro-2-methyl-5,6-dioxo-1,2,4-triazin-3-yl)thio)methyl) cephalosporanic acid monosodium salt (Compound Ia).

At 5° C., a three-neck flask was charged with Compound II (1 g), deionized water (4 mL), and 95% ethanol (4 mL). The mixture was agitated at 200 rpm to completely dissolve the solid. Sodium bicarbonate solution (8%, 1.8 mL) was dropwise added to the flask; after 30 minutes, the reaction mixture was discolored with activated carbon and then filtered. The filtration liquid was dropwise added to 100 mL of acetone to precipitate; The precipitation was monitored by an on-line particle analyzing instrument. The mixture was filtered and the solid was washed twice with acetone. Drying under vacuum at 40° C. for 24 hours gave 0.7 g of Compound Ia with a purity of 98.81% (HPLC).

¹HNMR (D₂O, 400 Hz): 1.25 (m, 12H, J=6.4), 3.45 (d, 1H, J=18), 3.69 (d, 1H, J=18), 3.62 (s, 3H), 3.98 (m, 4H), 4.07 (d, 1H, J=13.6), 4.32 (d, 1H, J=13.6), 5.12 (d, 1H, J=5), 5.56 (d, 1H, J=4.5).

### Example 4

1. The preparation of 7-(α-(((N,N′-diisopropylamidino)thio)acetylamino)-3-(((1,2,5,6-tetrahydro-2-methyl-5,6-dioxo-1,2,4-triazin-3-yl)thio)methyl) cephalosporanic acid (Compound II) is the same as in Example 1.

2. Preparation of 7-(α-(N,N′-diisopropylamidino)thio)acetylamino)-3-(((1,2,5,6-tetrahydro-2-methyl-5,6-dioxo-1,2,4-triazin-3-yl)thio)methyl) cephalosporanic acid monosodium salt (Compound Ia).

At 0° C., sodium carbonate (0.1 g) was dissolved in deionized water (5 mL) in a three-neck flask. Compound II (1 g) was added to the flask with agitation at 450 rpm, the pH value being 7.2, and the reaction was carried out for 45 minutes. The reaction mixture was discolored with activated carbon and filtered. The filtration liquid was dropwise added to 100 mL of ethanol to precipitate. The precipitation was monitored by an on-line particle analyzing instrument. The mixture was filtered and the solid was washed twice with acetone. Drying under vacuum at 40° C. yielded 0.65 g of Compound Ia with a purity of 98.35% (HPLC), pH 7.3. The ¹H-NMR results are the same as Example 2.

### Example 5

1. The preparation of 7-(α-((N,N′-diisopropylamidino)thio)acetylamino)-3-(((1,2,5,6-tetrahydro-2-methyl-5,6-dioxo-1,2,4-triazin-3-yl)thio)methyl) cephalosporanic acid (Compound II) is the same as in Example 1.

2. Preparation of 7-(α-((N,N′-diisopropylamidino)thio)acetylamino)-3-(((1,2,5,6-tetrahydro-2-methyl-5,6-dioxo-1,2,4-triazin-3-yl)thio)methyl) cephalosporanic acid monosodium salt (Compound Ia).

At 5° C., a three-neck flask was charged with Compound II (1 g), deionized water (4 mL), and 95% ethanol (4 mL). The mixture was agitated at 200 rpm to completely dissolve the solid. Sodium bicarbonate solution (8%, 1.8 mL) was dropwise added to the flask; after 30 minutes of reaction, the reaction mixture was discolored with activated carbon and filtered. The filtration liquid was dropwise added to 60 mL of acetone to precipitate. The precipitation was monitored by an on-line particle analyzing instrument. The mixture was filtered and the solid was washed twice with acetone. Drying under vacuum at 40° C. for 24 hours yielded 0.7 g of Compound Ia with a purity of 98.50% (HPLC). The ¹H-NMR results are the same as Example 2.

### Example 6

1. The preparation of 7-(α-((N,N′-diisopropylamidino)thio)acetylamino)-3-(((1,2,5,6-tetrahydro-2-methyl-5,6-dioxo-1,2,4-triazin-3-yl)thio)methyl) cephalosporanic acid (Compound II) is the same as in Example 1.

2. Preparation of 7-(α-((N,N′-diisopropylamidino)thio)acetylamino)-3-(((1,2,5,6-tetrahydro-2-methyl-5,6-dioxo-1,2,4-triazin-3-yl)thio)methyl) cephalosporanic acid monosodium salt (Compound Ia).

At 0° C., a three-neck flask was charged with deionized water (3 mL), acetonitrile (3 mL), and Compound II (1 g). The mixture was agitated at 250 rpm to completely dissolve the solid. Sodium hydroxide solution (2%, 3.5 mL) was dropwise added to the flask; after 30 minutes of agitation, the reaction mixture was discolored with activated carbon and filtered. The filtration liquid was drop-wise added to 100 mL of acetone to precipitate. The precipitation was monitored by an on-line particle analyzing instrument. The mixture was filtered and the solid was twice washed with acetone. Drying under vacuum (phosphorus pentoxide) at 40° C. yielded 0.65 g of Compound Ia with a purity of 97.12% (HPLC).

### Example 7

1. The preparation of 7-(α-((N,N′-diisopropylamidino)thio)acetylamino)-3-(((1,2,5,6-tetrahydro-2-methyl-5,6-dioxo-1,2,4-triazin-3-yl)thio)methyl) cephalosporanic acid (Compound II) is the same as in Example 1.

2. Preparation of disodium 7-(α-(N,N′-diisopropylamidino)thio)acetylamino)-3-(((1,2,5,6-tetrahydro-2-methyl-5,6-dioxo-1,2,4-triazin-3-yl)thio)methyl) cephalosporanate (Compound Ib).

The procedure is as Example 1, but 0.6 g, rather than 0.3 g, of sodium 2-ethylhexanoate was used. The product was dried under vacuum at 40° C. for 24 hours, yielding 0.76 g of Compound Ib with a purity of 96.72% (HPLC).

m/e: 616;

Elemental analysis: C₂₁H₂₇N₇Na₂O₆S₃

| | Calculated value | Measured value |
|---|---|---|
| C: | 40.93% | 41.11% |
| H: | 4.39% | 4.44% |
| N: | 15.92% | 16.01% |
| S: | 15.59% | 15.53% |

Sodium Ion Chromatography:

| | Calculated value | Measured value |
|---|---|---|
| Na: | 7.47% | 7.40% |

### Example 8

1. The preparation of 7-(α-((N,N′-diisopropylamidino)thio)acetylamino)-3-(((1,2,5,6-tetrahydro-2-methyl-5,6-dioxo-1,2,4-triazin-3-yl)thio)methyl) cephalosporanic acid (Compound II) is the same as in Example 1.

2. Preparation of disodium 7-(α-((N,N′-diisopropylamidino)thio)acetylamino)-3-(((1,2,5,6-tetrahydro-2-methyl-5,6-dioxo-1,2,4-triazin-3-yl)thio)methyl) cephalosporanate (Compound Ib).

The procedure is as Example 1, but 0.2 g, rather than 0.1 g, of sodium carbonate was used. The product was dried under vacuum at 40° C. for 24 hours, yielding 0.8 g of Compound Ib with a purity of 96.33% (HPLC), pH 10.02 (concentration 0.1 g/mL).

Sodium Ion Chromatography: C₂₁H₂₇N₇Na₂O₆S₃

| | Calculated value | Measured value |
|---|---|---|
| Na: | 7.47% | 7.59% |

### Example 9

1. The preparation of 7-(α-(N,N′-diisopropylamidino)thio)acetylamino)-3-(((1,2,5,6-tetrahydro-2-methyl-5,6-dioxo-1,2,4-triazin-3-yl)thio)methyl) cephalosporanic acid (Compound II) is the same as in Example 1.

2. Preparation of 7-(α-(N,N′-diisopropylamidino)thio)acetylamino)-3-(((1,2,5,6-tetrahydro-2-methyl-5,6-dioxo-1,2,4-triazin-3-yl)thio)methyl) cephalosporanic acid monopotassium salt (Compound Ic).

At 5° C., a three-neck flask was charged with deionized water (5 mL), Compound II (1 g), and potassium carbonate (0.15 g). The mixture was agitated at 400 rpm for 30 minutes, discolored with activated carbon, and filtered. The filtration liquid was dropwise added to 160 mL of acetone to precipitate. The precipitation was monitored by an on-line particle analyzing instrument; the mixture was filtered; the solid was twice washed with acetone. Drying under vacuum at 40° C. for 24 hours yielded 0.73 g of Compound Ic with a purity of 97.43% (HPLC).

Elemental Analysis: C₂₁H₂₈KN₇O₆S₃

| | Calculated value | Measured value |
|---|---|---|
| C: | 41.33% | 41.1% |
| H: | 4.59% | 4.65% |
| N: | 16.07% | 16.01% |
| S: | 15.75% | 15.70% |

Potassium Ion Chromatography:

| | Calculated value | Measured value |
|---|---|---|
| K: | 6.41% | 6.64% |

### Example 10

1. The preparation of 7-(α-((N,N′-diisopropylamidino)thio)acetylamino)-3-(((1,2,5,6-tetrahydro-2-methyl-5,6-dioxo-1,2,4-triazin-3-yl)thio)methyl) cephalosporanic acid (Compound II) is the same as in Example 1.

2. Preparation of 7-(α-((N,N-diisopropylamidino)thio)acetylamino)-3-(((1,2,5,6-tetrahydro-2-methyl-5,6-dioxo-1,2,4-triazin-3-yl)thio)methyl) cephalosporanic acid monopotassium salt (Compound Ic).

At 5° C., a three-neck flask was charged with deionized water (5 mL), Compound II (1 g), and 95% ethanol (5 mL). After the solid was completely dissolved, Potassium bicarbonate (0.17 g) was added to the flask at 400 rpm agitation for 30 minutes; the reaction mixture was discolored with activated carbon and filtered. The filtration liquid was dropwise added to 160 mL of acetone to precipitate. The precipitation was monitored by an on-line particle analyzing instrument. The mixture was filtered and the solid was twice washed with acetone. Drying under vacuum at 40° C. for 24 hours yielded 0.67 g of Compound Ic with a purity of 97.1% (HPLC).

Potassium Ion Chromatography:

| | Calculated value | Measured value |
|---|---|---|
| K: | 6.41% | 6.29% |

### Example 11

1. The preparation of 7-(α-((N,N′-diisopropylamidino)thio)acetylamino)-3-(((1,2,5,6-tetrahydro-2-methyl-5,6-dioxo-1,2,4-triazin-3-yl)thio)methyl) cephalosporanic acid (Compound II) is the same as in Example 1.

2. Preparation of dipotassium 7-(α-((N,N′-diisopropylamidino)thio)acetylamino)-3-(((1,2,5,6-tetrahydro-2-methyl-5,6-dioxo-1,2,4-triazin-3-yl)thio)methyl) cephalosporanate (Compound Id).

At 0° C., a three-neck flask was charged with deionized water (5 mL), Compound II (1 g), and potassium carbonate (0.3 g). The mixture was agitated at 350 rpm for 45 minutes; the reaction mixture was discolored with activated carbon and filtered. The filtration liquid was dropwise added to 200 mL of acetone to precipitate white solid. The precipitation was monitored by an on-line particle analyzing instrument. The mixture was filtered and the solid was twice washed with acetone. Drying under vacuum at 40° C. for 24 hours yielded 0.85 g of Compound Id with a purity of 97.03% (HPLC).

Elemental analysis: C₂₁H₂₇K₂N₇O₆S₃

| | Calculated value | Measured value |
|---|---|---|
| C: | 38.90% | 39.1% |
| H: | 4.17% | 4.35% |
| N: | 15.13% | 15.22% |
| S: | 14.82% | 15.07% |

Potassium Ion Chromatography:

| | Calculated value | Measured value |
|---|---|---|
| K: | 12.04% | 12.13% |

### Example 12

### Preparation of Antibiotic Drug

1. The preparation of 7-(α-((N,N′-diisopropylamidino)thio)acetylamino)-3-(((1,2,5,6-tetrahydro-2-methyl-5,6-dioxo-1,2,4-triazin-3-yl)thio)methyl) cephalosporanic acid (Compound II) is the same as in Example 1.

2. At 5° C., a three-neck flask was charged with Compound II (100 g), injection water (400 mL), and 95% ethanol (400 mL) with agitation (200 rpm). After the solid was completely dissolved, sodium bicarbonate solution (8%, 180 mL) was dropwise added to the flask. After 30 minutes of reaction, the reaction mixture was discolored with injection-grade activated carbon and filtered with 0.45 μM membrane. In a clean environment, the filtration liquid was dropwise added to 10 L of acetone to precipitate. The precipitation was monitored by an on-line particle analyzing instrument. The mixture was filtered and the solid was twice washed with acetone. Drying under vacuum at 40° C. for 24 hours yielded 68 g of Compound Ia. The compound was grinded into powder and measured for purity and water content. It was packed under sterile conditions into bottles with 0.5 g/bottle or 1.0 g/bottle and capped.

### Example 13

### Preparation of Tablets

### Formulation:

| | Per tablet | Per 1000 tablet |
|---|---|---|
| Compound | 250 mg | 250 g |
| Lactose | 63.6 mg | 63.6 g |
| Gelatinized starch | 81.5 mg | 81.5 g |
| Magnesium stearate | 4.1 mg | 4.1 g |

Preparation: Compound Ia from Example 12, lactose, and gelatinized starch were mixed for 15-30 minutes and then mixed with 10% PVP adhesive solution. The mixture was made into particles. The particles were dried at 50° C. for 2 hours, shipped, and then thoroughly mixed with magnesium stearate. The mixture was compressed into tablets. The tablets were covered with 10% HDMC solution film and packaged.

What is claimed is:

1. A N-heterocyclic substituent-containing antibiotic comprising sodium or potassium salts of 7-(α-((N,N′-diisopropylamidino)thio)acetylamino)-3-(((1,2,5,6-tetrahydro-2-methyl-5,6-dioxo-1,2,4-triazin-3-yl)thio)methyl) cephalosporanic acid having the general structure (I): 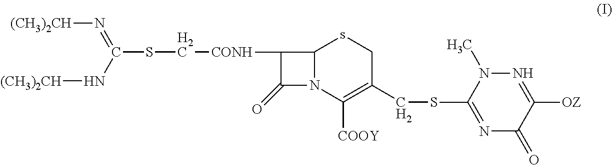
wherein:
Ia: Y=H, Z=Na;
Ib: Y=Na, Z=Na;
Ic: Y=H, Z=K;
Id: Y=K, Z=K.

2. A method for preparing a N-heterocyclic substituent-containing antibiotic comprising the sodium or potassium salts of 7-(α-((N,N′-diisopropylamidino)thio)acetylamino)-3-(1,2,5,6-tetrahydro-2-methyl-5,6-dioxo-1,2,4-triazin-3-yl)thio)methyl) cephalosporanic acid, said method comprising:
(a) dissolving or suspending 7-(α-((N,N′-diisopropylamidino)thio)acetylamino)-3-(((1,2,5,6-tetrahydro-2-methyl-5,6-dioxo-1,2,4-triazin-3-yl)thio)methyl) cephalosporanic acid into a salt forming solvent system and reacting therein with a sodium-containing or potassium-containing alkali compound; and
(b) precipitating the product in a precipitation agent and isolating the product.

3. The method of claim 2, wherein the salt forming solvent system is selected from the group consisting of water, alkyl ketones, alcohols, nitriles, amides, and mixtures thereof;
and wherein the alkyl group is a C₁-C₁₂, preferably C₁-C₄, alkyl group.

4. The method of claim 2, wherein the sodium-containing alkali compound is selected from C₁-C₅ sodium alkoxides, sodium aliphatic or aromatic carboxylates, or inorganic sodium salts including sodium methoxide, sodium acetate, sodium 2-ethylhexanoate, sodium tartrate, sodium hydroxide, sodium carbonate, and sodium bicarbonate; and the potassium-containing alkali compound is selected from potassium hydroxide, potassium carbonate, and potassium bicarbonate.

5. The method of claim 2, wherein the precipitation agent is selected from C₁-C₄ alcohols, C₃-C₆ ketones, or mixtures thereof.

6. The method of claim 2, wherein the molar ratio of the sodium-containing or potassium-containing alkali compound to 7-(α-((N,N′-diisopropylamidino)thio)acetylamino)-3-(((1,2,5,6-tetrahydro-2-methyl-5,6-dioxo-1,2,4-triazin-3-yl)thio)methyl) cephalosporanic acid is 1:1 for forming its monosodium salt or mono potassium salt; or the ratio is 2:1 for forming its disodium salt or dipotassium salt.

7. The N-heterocyclic substituent-containing antibiotic of claim 1, which further comprises a vehicle for a drug.

8. An anti-infective drug comprising the N-heterocyclic substituent-containing antibiotic of claim 1.
